(19) United States
(12) Patent Application Publication
(10) Pub. No.: US 2004/0197291 A1
(43) Pub. Date: Oct. 7, 2004

(54) Skin-tightening preparations containing gliadin

(75) Inventors: Dharman Makwana, Platte City, MO (US); Clodualdo C. Maningat, Platte City, MO (US); Sukh Bassi, Atchison, KS (US); Jeremy T. Miller, Platte City, MO (US)

Correspondence Address: HOVEY WILLIAMS LLP, 2405 GRAND BLVD., SUITE 400, KANSAS CITY, MO, 64108, US

(73) Assignee: MGP INGREDIENTS, INC.

(21) Appl. No.: 10/404,585

(22) Filed: Apr. 1, 2003

(51) Int. Cl. A61K 7/06; A61K 7/11

(52) U.S. Cl. 424/70.14

(57) ABSTRACT

The present invention is concerned with preparations for application to human skin including a cream or lotion base together with a quantity of a skin-tightening agent consisting essentially of gliadin. Products such as creams, lotions, facial masks and sunscreens can be prepared which exhibit desirable skin-tightening or anti-wrinkle effects.

### BACKGROUND OF THE INVENTION

1. Field of the Invention

The present invention is broadly concerned with cosmetic-type preparations having a skin-tightening or anti-wrinkle effect. More particularly, the invention is concerned with such preparations which include a cream or lotion base typically having respective quantities of oil, emulsifier, thickener and water, together with a skin-tightening agent consisting essentially of gliadin. Preparations in accordance with the invention have been shown to exhibit significant skin-tightening effects.

2. Description of the Proir Art

A vast number of cosmetic or similar preparations have been developed in the past in the form of creams or lotions. There is considerable variation in the makeup of these formulations depending upon the intended effect. U.S. Pat. No. 5,780,013 describes gliadin-containing hairsprays having low volatile organic compound (VOC) levels. Similarly, U.S. Pat. No. 5,945,086 discloses a number of cosmetic formulations including gliadin, e.g., shampoos, conditioners, styling gels, sunscreens, shaving creams and bath and shower gels. U.S. Pat. No. 4,518,614 is directed to cosmetic preparations of the liquid or emulsion type including minor amounts of gibberellic acid and lysine in order to soften and improve the texture of skin, moisturize the epidermis and diminish skin wrinkles. The '614 reference also indicates that the formulations containing gibberellic acid and lysine may also be supplemented with minor amounts of gliadin.

Gliadin is a monomeric protein having an average molecular weight of about 30,000-50,000 daltons. Gliadin can be obtained by fractionation of wheat gluten and is considered to be a premium product. Gliadin is known to improve the freeze thaw stability of frozen doughs and also improve microwave stability. The product may also be used as a chewing gum base replacer, a pharmaceutical binder, and to improve the texture and mouth feel of pasta products; although gliadin has also been used in certain cosmetic products, it has never found utility in hairsprays or similar compositions.

### SUMMARY OF THE INVENTION

The present invention is concerned with improved preparations for application to skin in order to provide a significant skin-tightening and anti-wrinkle effect. Broadly, the preparations of the invention include a cream or lotion base which may contain a variety of specific ingredients, together with a quantity of a skin-tightening agent consisting essentially of gliadin. It has been discovered that gliadin itself provides a desirable skin-tightening effect in cosmetic-type preparations, without the addition of ingredients such as gibberellic acid and lysine as required by the above-described Pat. No. 4,518,614.

In more detail, the base of the preparations typically include respective quantities of oil, emulsifier, thickener and water, generally at levels of from about 2-6% by weight oil, from about 3-10% by weight emulsifier, from about 0.01-4% by weight thickener, and from about 65-90% by weight water. More preferred bases include oil, from about 3-5% by weight oil, from about 5-7% by weight emulsifier, from about 0.1-2% by weight thickener and from about 70-80% by weight water. The final preparations of the invention should normally have a Brookfield viscosity of from about 20,000-50,000 cps using a TB spindle at 25° C. and 5 rpm.

The skin-tightening agent used in the preparation of the invention consists essentially of gliadin, which is normally present at a level of from about 0.5-7% by weight, and more preferably from about 1.5-5% by weight. The most preferred gliadin is highly purified wheat-derived gliadin.

The preparations of the invention may also include a variety of other base ingredients such as those selected from the group consisting of humectants, emollients, skin conditioning agents, sunscreen agents, pH adjustment agents, fragrances and antibacterial components. These are normally used at conventional, art-recognized levels.

A number of cosmetic or cosmetic-type preparations can be prepared in accordance with the invention. Thus, preparations selected from the group consisting of skin creams, facial mask, shave creams and sunscreens can all be readily formulated. The use of such products involves application thereof to the skin, and the most beneficial results are achieved with creams or the like designed to be placed on the skin for extended periods, of at least 2 hours and more preferably for at least 4 hours.

### BRIEF DESCRIPTION OF THE DRAWINGS

FIG. 1 is a schematic representation of test apparatus used to determine the skin-tightening effects of compositions in accordance with the invention;

FIG. 2 is a plot of force versus time, depicting the results of a skin-tightening test using a gliadin-containing preparation in accordance with the invention; and

FIG. 3 is a plot of force versus time similar to that of FIG. 2, but illustrating the effects using a control preparation free of gliadin.

### DETAILED DESCRIPTION OF THE PREFERRED EMBODIMENT

The following examples set forth preferred skin-tightening in accordance with the invention, as well as a technique for determining the skin-tightening effects thereof. It is to be understood, however, that these examples are provided by way of illustration and nothing therein should be taken as a limitation upon the overall scope of the invention.

### EXAMPLE 1

A skin cream was prepared containing the following ingredients:

TABLE 1
Skin Cream

| Phase | Trade Name | INCI Name | Function | % W/W |
|---|---|---|---|---|
| A | Distilled Water | Distilled Water | Aqua | QS |
| A | Glycerin | Glycerin USP | Humectant | 2.0 |
| A | Carbomer | Carbomer 940 | Thickener | 0.10 |
| B | Stepan DGS SE | Triple Press Stearic Acid | Emulsifier | 3.00 |
| B | Lipocol C | Cetyl Alcohol | Emulsifier | 2.00 |
| B | Lipo GMS-450 | Glyceryl Stearate | Emulsifier | 2.00 |
| B | Promulgin D | Cetyl Alcohol Ceteareth-20 | Emulsifier | 1.20 |
| B | Coconut Oil | Coconut Oil | Emollient | 0.50 |
| B | Lipo IPP | Isopropyl Myristate | Emollient | 0.20 |
| B | Lipo IPM | Isopropyl Palmitate | Emollient | 0.50 |
| B | Lipowax G | Stearyl Alcohol | Emulsifier | 0.75 |
| B | Jojoba oil | Jojoba Oil White | Emollient | 0.30 |
| C | Triethanolamine 99% | Triethanolamine 99% | pH Adjuster | QS |
| C | Dow Corning 200-350 ct | Dimethicone | Feel | 0.20 |
| D | Fragrance | Fragrance | Perfume | 0.05 |
| B | Preservative | QS | Antibacterial | QS |
| F | Omni-Smooth | Wheat Gliadin | Anti-Wrinkle | 3.00 |

The ingredients of Phase A were first placed in a suitable primary tank by first adding the distilled water and then the remaining ingredients; the Phase A mixture was then heated to 75° C. and mixed to insure that all of the Carbomer was in solution. The ingredients of Phase B were then weighed into a secondary tank and heated to 75° C. The Phase B mixture was then added to the Phase A mixture at 75° C. The Phase A/Phase B mixture was then cooled and at 50-55° C., the ingredients of Phase C were added. When the temperature reached 35° C., the ingredients of Phase D and E were added. When the temperature reached 25° C., the Phase F gliadin was sifted into the mixture slowly with mixing, resulting in a smooth cream.

As a comparison, a skin cream identical with that of Table 1 was prepared except that no Phase F gliadin was added.

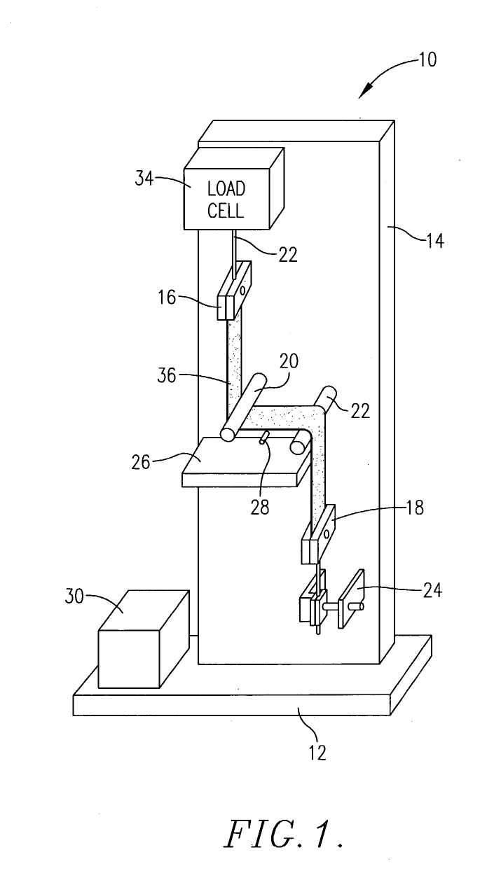

In order to test the skin-tightening effects of the gliadin-containing composition versus the no-gliadin control, a device 10 of the type schematically illustrated in FIG. 1 was employed. The device 10 included a cross-head 12 of the type found on typical Instron equipment with an upstanding standard 14 secured to cross-head 12. A pair of skin-holding clamps 16 and 18 are supported on standard 14, along with a pair of intermediate rollers 20, 22 and a stationary lower clamp 24. A heating block 26 is located below the rollers 20, 22 as shown along with a thermocouple 28. An electronic temperature controller 30 is supported on cross-head 12 and is operatively connected to block 26 and thermocouple 28 in order to provide controlled heating. Finally, a probe 32 associated with clamp 16 is coupled with a conventional load cell 34. The device 10 is designed to hold a length of vitro skin 36 for test purposes.

In more detail, the comparative test of the above-described preparations involved providing a strip of synthetic skin (9.5 cm×2.0 cm) looped over the rollers 20, 22 and held in place via clamps 16, 18 and 24. In this instance, the synthetic skin was obtained from IMS, Inc., Milford, Conn. and was used after overnight equilibration at 65% relative humidity and 21° C. ambient temperature. The active area of the strip was approximately 2.5 cm×2.0 cm, at the region between rollers 20, 22. The temperature controller 30, heating block 26 and thermocouple 28 were employed to maintain the temperature of the strip between the rollers at approximately body temperature, 38° C.

Initially, a small tension was applied to the substrate creating a small load cell output. When this force leveled off, the respective preparations were applied and the increase in load cell output (due to contractile forces, if any) was recorded as a function of time. Three replicates were carried out using the gliadin-containing preparation and the control preparation.

Each of the test preparations softened the substrate and therefore a reduction in the load cell output was initially observed. In the control preparation, the force, after the initial decrease, increased and leveled off within the next 3 hours. The replicate measurements demonstrated that the final force was smaller than the initial force indicating softening of the substrate upon application of the control preparation. After application of the gliadin-containing preparation, the highest contractile forces were obtained within 2-3 hours, being 60-70 g higher as compared with the initial force. Then, the force decreased and remained constant for at least the next 8 hours. The difference between the final force after 8 hours and the initial force before application of the gliadin-containing preparation were positive in all three replications (between 15-30 g). This confirms the contraction of the vitro skin. Table 2 below sets forth the averages of these initial and post-application forces for the three replications, and the data represents an averaging of many hundreds of data points.

TABLE 2
Average contractile forces for control and
active containing formulations

| | | | | Change in |
|---|---|---|---|---|
| Formulation | Test No. | Initial Force (g) | Final Force (g) | Force (g) |
| DM-A Control | 1 | 55.0 | 15.0 | (−) 40.0 |
| | 2 | 60.0 | 40.0 | (−) 20.0 |
| | 3 | 60.0 | 45.0 | (−) 15.0 |
| DM-B Active | 1 | 40.0 | 70.0 | (+) 30.0 |
| | 2 | 100.0 | 115.0 | (+) 15.0 |
| | 3 | 110.0 | 140.0 | (+) 30.0 |

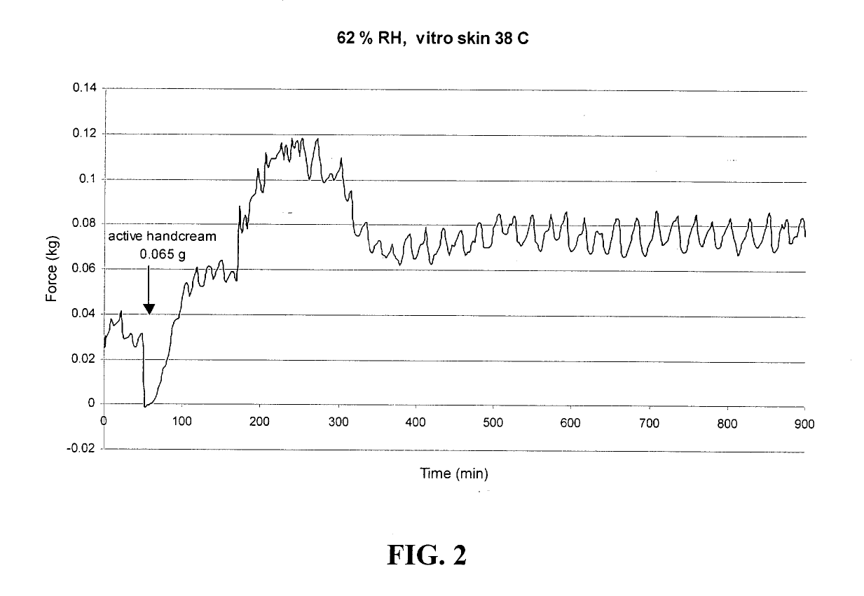

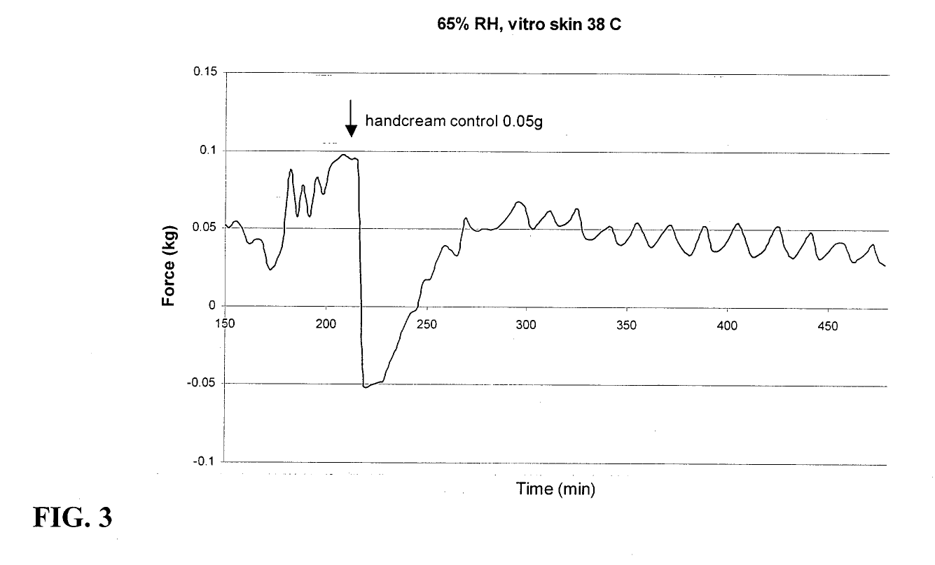

FIGS. 2 and 3 depict typical force curves obtained for the active and control creams, respectively. FIG. 2 clearly shows a positive difference between the final force after 8 hours and the initial force before cream application, whereas FIG. 3 shows that after cream application, the contractile forces of the skin never regained its pre-application level.

In summary, these tests demonstrated a pronounced skin-tightening produced as a result of the gliadin-containing preparation onto the synthetic vitro skin held at body temperature, with the maximum effect achieved after about 5-6 hours. Such contractile forces are not seen using the no-gliadin control.

### EXAMPLE 2

In this example, a smooth shave cream was prepared containing the following ingredients.

TABLE 3
Smooth Shave Cream

| Phase | Ingredient | % W/W |
|---|---|---|
| A | Deionized Water | Adjust |
| A | Lauric Acid | 1.50 |
| B | Stearic Acid | 15.00 |
| B | Coconut Fatty Acid | 2.50 |
| C | Sodium Hydroxide | 0.40 |
| C | Potassium Hydroxide | 4.00 |
| D | Omni-Smooth (Gliadin) | 0.50 |
| D | Foam Pro L (Hydrolyzed | 1.00 |
| | Wheat Protein) | |
| D | Glycerin | 7.00 |
| E | Fragrance | QS |
| E | Preservative | QS |
| F | SD-40 | 10.00 |

The ingredients of Phase A were initially mixed and heated to 75° C. The ingredients of Phase B were separately heated at this same temperature and mixed with Phase A while mixing. The ingredients of Phase C were premixed in 20% deionized water and added to the Phase A/Phase B mixture while mixing. The resulting mixture was cooled to 35° C. and a premix of the Phase D ingredients was added along with the ingredients of Phase E. Finally, at 25° C., the Phase F ingredient was added.

### EXAMPLE 3

In this instance, a facial mask was prepared having the ingredients set forth in Table 4.

TABLE 4
Facial Mask

| Phase | Trade Name | INCI Name | Amount | Function |
|---|---|---|---|---|
| A | Aqua | Distilled Water | QS | QS |
| A | Versene NA | Disodium EDTA | 0.10 | Chelation |
| A | Stepanol CA-330 | Ammonium Laureth Sulfate | 2.00 | Cleaning |
| A. | Carbopol 940 | Carbomer | 0.16 | Thickener |
| A | Triethanolamine (99%) | Triethanolamine (99%) | QS | pH Adjust |
| B | Almond Oil | Almond Oil | 1.50 | Emollient |
| B | Jojoba Oil | Jojoba Oil Golden | 2.00 | Emollient |
| B | Lipocol S | Stearic Acid | 2.00 | Emulsifier |
| B | Lipocol GMS-450 | Glyceryl Stearate | 1.50 | Emulsifier |
| C | TiO2 | Titanium Dioxide | 5.00 | Colorant |
| C | Bentonite | Bentonite 670 | 3.50 | Bulking |
| C | Kaoline | Kaoline Colloidal NF | 5.00 | Bulking |
| D | Preservative | Preservative | QS | Antibacterial |
| D | Yellow #5 | FD&C Yellow #5 | QS | Color |
| D | Blue #01 | FD&C Blue #1 | QS | Color |
| D | Fragrance | Fragrance | QS | Fragrance |
| E | Glycerin | Glycerin | 4.00 | Humectant |
| E | Aqua | Distilled water | 20.00 | QS |
| E | Omni-Smooth | Gliadin | 3.00 | Anti-Wrinkle |

In preparative procedures, the ingredients of Phase A were mixed in a primary tank and heated to 75° C. to insure that all of the Carbomer was in solution. All of the ingredients of Phase B were mixed in a secondary tank and heated to 75° C. Phase B was then added to Phase A with continued mixing. The Phase C ingredients were then to the Phase A/Phase B mixture. The resultant mixture was cooled and at 35° C., the ingredients of Phase D were added with mixing. The ingredients of Phase E were premixed and added to the batch at 25° C. The mask is used by applying to the face and neck avoiding the eye and lip areas. After 10-15 minutes, the mask is rinsed and removed with warm water.

### EXAMPLE 4

In this example, an anti-wrinkle sunscreen was prepared containing the ingredients of Table 5.

TABLE 5
Anti-Wrinkle Sunscreen

| Phase | Trade Name | INCI Name | Function | % W/W |
|---|---|---|---|---|
| A | Distilled Water | Distilled Water | Aqua | QS |
| A | Glycerin | Glycerin USP | Humectant | 2.00 |
| A | Carbomer | Carbomer 940 | Thickener | 0.25 |
| B | Stepan DGS SE | Triple Press Stearic Acid | Emulsifier | 3.00 |
| B | Lipocol C | Cetyl Alcohol | Emulsifier | 1.50 |
| B | Lipo GMS-450 | Glyceryl Stearate | Emulsifier | 2.50 |
| B | Promulgin D | Cetyl Alcohol Ceteareth-20 | Emulsifier | 1.50 |
| B | Liponate CG | Caprylic/Capric Triglyceride | Skin Conditioning | 2.00 |
| B | Parasol 340 | Octocryleneyristate | Sunscreen Agent | 3.50 |
| B | Benophenome-3 | Uvinul - 40 | Sunscreen Agent | 0.05 |
| C | AMP | 2-amino-2-methyl-1-propanol | pH Adjuster | QS |
| C | Dow Corning 200-350 ct | Dimethicone | Feel | 1.50 |
| D | Dow Corning Fluid 244 | Cyclomethicone | Feel | 3.00 |
| D | Fragrance | Fragrance | Perfume | 0.05 |
| E | Preservative | QS | Antibacterial | QS |
| F | Omni-Smooth | Gliadin | Anti-Wrinkle | 3.00 |

The sunscreen was prepared by first adding the distilled water to a primary tank followed by addition of the Phase A ingredients and heating to 75° C. with mixing until all of the Carbomer was in solution. The ingredients was Phase B were then added to a secondary tank and heated to 75° C. The Phase A/Phase B mixture was then cooled and mixed, and at 50-55° C., the ingredients of Phase C were added. With further cooling and mixing, the ingredients of Phases D and E were added at 35° C. Finally, at 25° C., the gliadin was slowly added until a smooth cream preparation was obtained.

We claim:

1. In a preparation for application to skin comprising a cream or lotion base, the improvement which comprises a quantity of a skin-tightening agent consisting essentially of gliadin.

2. The preparation of claim 1, said base comprising respective quantities of oil, emulsifier, thickener and water.

3. The preparation of claim 2, said oil being present at a level of from about 2-6% by weight, said emulsifier present at a level of from about 3-10% by weight, said thickener present at a level of from about 0.01-4% by weight, and said water present at a level of from about 65-90% by weight.

4. The preparation of claim 1, said preparation having a Brookfield viscosity of from about 20,000-50,000 cps using a TB spindle at 25° C. and 5 rpm.

5. The preparation of claim 1, said gliadin being present at a level of from about 0.5-7% by weight.

6. The preparation of claim 5, said level being from about 1.5-5% by weight.

7. The preparation of claim 1, said base further including an ingredient selected from the group consisting of humectants, emollients, skin conditioning agents, sunscreen agents, pH adjustment agents, fragrances and antibacterial components.

8. The preparation of claim 1, said preparation selected from the group consisting of skin creams, facial mask, shave creams and sunscreens.

9. A method of tightening skin comprising the step of applying to the skin a quantity of a preparation in accordance with claim 1.
